(19) United States
(12) Patent Application Publication
(10) Pub. No.: US 2010/0279353 A1
(43) Pub. Date: Nov. 4, 2010

(54) Process of producing fibrinolytic enzyme from mushroom

(76) Inventors: Shiu Nan Chen, San Marino, CA (US); Chung Lun Lu, San Marino, CA (US); Jung Fu Wu, San Marcos, CA (US); Sherwin Chen, San Marino, CA (US)

Correspondence Address: DAVID AND RAYMOND PATENT FIRM, 108 N. YNEZ AVE., SUITE 128, MONTEREY PARK, CA, 91754, US

(21) Appl. No.: 12/387,340

(22) Filed: Apr. 30, 2009

(51) Int. Cl. C12P 21/04; C12N 9/02

(52) U.S. Cl. 435/70.1; 435/189

(57) ABSTRACT

A fibrinolytic enzyme isolated from a culture broth of a mushroom has a characteristic of degrading a fibrin and a fibrinogen without activating an activity of a plasminogen. The plasminogen is activated to generate a plasmin to degrade the fibrin and/or fibrinogen, so that the fibrinolytic enzyme be used for the thrombosis-related diseases to degrade the fibrin and fibrinogen of blood clots without activate the plasminogen, so as to avoid a hemorrhage due to the over activation the plasminogen to over generate the plasmin.

### BACKGROUND OF THE PRESENT INVENTION

1. Field of Invention

The present invention relates to an enzyme, and more particularly to a process for producing a fibrinolytic enzyme ScFz, wherein the fibrinolytic enzyme is capable of degrading fibrin and fibrinogen without activating the plasminogen to plasmin, so that the fibrinolytic enzyme can be widely applied on the fields of biotechnology, medicine, or medical development, such as thrombosis therapies, to avoid unexpected hemorrhage.

2. Description of Related Arts

When a vascular is damaged, the inner surface of the blood vessel is exposed and the platelets are activated to form a blood clot at the wound to temporarily stop the blood bleeding from the blood vessel and against the arterial pressure inside the vessel so as to recover the wound. The blood clot is mainly composed of a primary structure protein of fibrin, wherein a thrombin, which is one of the catalysts of enzymes, converts a fibrinogen into the fibrin. The stability of the blood clot such as blood clot retraction and fibrinolytic properties, are affected by the structural, biological, physical, and chemical properties occurred during the formation process of the blood clot.

In a balance environment of physiological human body, plasmin will degrade the blood clots to avoid the thrombosis in the vascular vessel. If the blood clots can not be. degraded, the blood clots become the thrombus to decrease the blood flow through that vessel. This may be resulting in death of tissue supplied by that vessel, such as cardiovascular disease or hypertension.

At present, the medications treatments of the thrombosis related diseases include anticoagulant, anti-platelet aggregation agent, and thrombolytic agents. According to the mechanism process, the thrombolytic agents can be classified into two types. The first type thrombolytic agents activate the plasminogen to be converted into the plasmin, so that the plasmin can further degrade the fibrin so as to thrombolysis the blood clots or thrombus, such as the tissue-type plasminogen activator (t-PA), urokinase, streptokinase etc.(Collen and Lijnen 2004, Duffy 2002). The second type thrombolytic agents can directly degrade the fibrin, such as the Human fibrinolysin, lumbrokinase etc. Many of the thrombolytic agents are extracted from animals such as snakes (Giron et al., 2008) and earthworms (Ge et al., 2005; Park et al., 1998), bacteria such as Bacilli subtilis (Omura et al., 2005; Ko et al., 2004; Choi et al., 2004) and Streptomyces megasporus (Chitte and Dey 2000), fungi (Kim and Kim, 1999; Choi and Sa, 2000; Kim and Kim, 2001; Lee et al., 2005; Hattori et al., 2005; Park et al., 2007; Li et al., 2007), and seaweed (Matsubara et al., 1999) etc.

According to the above first type thrombolytic agent, it is to convert the plasminogen into the plasmin to accomplish degrading the thrombus. Therefore, to treat the thrombosis related diseases with the first type thrombolytic agent may cause the risk of over producing the plasmin to result in the hemorrhage danger. Moreover, the lumbrokinase of the second type thrombolytic agent is actually a mixture. It not only has the ingredient for degrading the fibrin, but also has the ingredient for activating the plasminogen. This has the same problem as the first type thrombolytic agent which may result in the hemorrhage danger. Therefore, clinically there is a need of an agent which can degrade the fibrin directly without activating the plasminogen so as to prevent producing excessive plasmin to cause the hemorrhage.

On the other hand, no matters in Asia or western countries, gill fungus or mushrooms are to be used as food or medicine (Sullivan et al., 2006). Gill fungus or mushrooms are known to contain a variety of activated proteins or peptides. Such activated proteins and peptides are studied of their effectiveness in a human body (Ng, 2004). Furthermore, traditionally Schizophyllum commune is used for medication purpose. Currently known that a long chain macromolecule polysaccharide molecule from the fermentation submerged culture of Schizophyllum commune called schizophyllan, shows many pharmacological activities including anti-tumor effect and immunology enhancement (Kumari et al., 2008). At present, there's no studies show any ingredients isolated from the Schizophyllum commune can degrade the blood fibrin and does not activate the plasminogen to convert into plasmin.

### SUMMARY OF THE PRESENT INVENTION

A main object of the present invention is to provide a fibrinolytic enzyme, wherein the fibrinolytic enzyme can degrade the fibrin or the fibrinogen directly and without activating the plasminogen to generate plasmin, so as to avoid over producing the plasmin to cause the danger of hemorrhage.

Another object of the present invention is to provide a fibrinolytic enzyme which can be used for the treatment of the thrombosis-related disease, wherein the ScFz can avoid the hemorrhage side effect of the conventional thrombolytic agent due to the over activating the plasminogen.

Another object of the present invention is to provide a fibrinolytic enzyme for a medication, wherein the medication comprises the novel fibrinolytic enzyme and a clinical acceptable carrier for carry the novel fibrinolytic enzyme and other ingredients for medical purpose.

Another object of the present invention is to provide a fibrinolytic enzyme as a thrombolytic agent, wherein the thrombolytic agent comprises the novel fibrinolytic enzyme being used for the treatment of thrombosis-related disease and avoid the risk of hemorrhage danger of the conventional thrombolytic agent.

Another object of the present invention is to provide a fibrinolytic enzyme, wherein the fibrinolytic enzyme is isolated from a mushroom, preferable from an edible or medicinal mushroom, and more preferable from a Schizophyllum commune of mushroom.

Another object of the present invention is to provide a fibrinolytic enzyme, wherein the fibrinolytic enzyme from the Schizophyllum commune is a monomer having the molecular weight 20˜23 kD.

Another object of the present invention is to provide a method of preparation a fibrinolytic enzyme, wherein an edible or medicinal mushroom is adapted for isolating the plasmin.

Another object of the present invention is to provide a method of preparation the fibrinolytic enzyme, wherein the mushroom adapted for isolating the fibrinolytic enzyme is cultured in the same way as the conventional culture way of edible or medicinal mushroom.

Another object of the present invention is to provide a method of preparation the fibrinolytic enzyme, wherein, comparing to the prior arts of heating extraction or organic solvent extraction method, the method of the present invention adapts a filtration and chromatography ways for isolating the novel fibrinolytic enzyme from mushroom without heating or adding any organic solvents, so that the method can effectively retain the activity of the fibrinolytic enzyme.

Accordingly, in order to accomplish the above objects, the fibrinolytic enzyme of the present invention, ScFz, has the characteristics of:

(a) having an amino acid N-terminus sequence of SEQ ID NO.1, ASYNGXSS, wherein A is alanine, S is serine, Y is tyrosine, N is asparagines, G is glycine, and X is undetermined;

(b) having partial protein fragment the same as the gil81175178 protein fragment showed in LC/MS/MS mass spectrographic analysis; and

(c) having a molecular weight around 20 to 23 kDa, and more particularly 21.32 kDa.

Accordingly, in order to accomplish the above objects, the method of preparation the fibrinolytic enzyme comprises the steps of the followings:

(a) culturing a mushroom by fermentation in a culture broth;

(b) removing a mycelium of the mushroom from the above culture broth by filtration;

(c) separating molecules of the culture broth with different pore size filtration by cross-flow filtration system using a ceramic columns, wherein the culture broth is is separated into a low molecular weight (MW) solution and a high molecular weight (MW) solution;

(d) precipitating a crude protein from the low molecular weight solution by adding an ammonium sulfate and remove salt in the crude protein by dialysis; and

(e) purifying the crude protein from the step (d) to get a fibrinolytic enzyme.

These and other objectives, features, and advantages of the present invention will become apparent from the following detailed description, the accompanying drawings, and the appended claims.

### BRIEF DESCRIPTION OF THE DRAWINGS

FIGS. 1A, 1B, 1C and 1D show the results of the purifying step of fibrinolytic enzyme, ScFz, from a Schizophyllum commune culture broth, wherein the purifying step includes three sub-steps.

FIG. 1A shows the chromatography of the first sub-step purification of ScFz and a protease activity of fractions thereof, wherein the ScFz is separated by a hydrophobic interaction chromatography on Phenyl Sepharose™ High Performance beaded packing column. The high protease activity fractions (No. 25-28) is pooled, precipitated, dialyzed and applied onto the next step.

FIG. 1B shows the chromatography of the second sub-step purification of ScFz and the protease activity of fractions thereof, wherein the ScFz is separated by a strong anion exchange chromatography on Mono Q column. The high protease activity fractions (No. 17-21) is pooled, precipitated, dialyzed and applied onto the next step.

FIG. 1C shows the chromatography of the third sub-step purification of ScFz and the protease activity of fractions thereof, wherein the ScFz is separated by a gel filtration on Superdex 75 10/300 GL column. The ScFz is obtained in fraction No. 5.

FIG. 1D is the fractions collected from the hydrophobic interaction chromatography examined by an artificial fibrin plate assay, illustrating the + is a Human plasmin 10 μg as a positive control. The clear rings are indicating the positive results for the fractions.

FIGS. 2A and 2B show the result of the Molecular weight (M.W.) determination for fibrinolytic enzyme, ScFz, from the S. commune culture broth.

FIG. 2A is the Semi-logarithmic plot of the protein marker flow through size-exclusion gel filtration column, comprising a ribonuclease A (13.5 kDa), carbonic anhydrase (29 kDa), apoferritin (44.3 kDa) and bovine serum albumin (67 kDa), wherein the black arrow shows the M.W. of ScFz is 21.33 kDa.

FIG. 2B is the SDS-polyacrylamide gel (11%) electrophoresis for each purifying step; M: Marker. Lane 1: Crude proteins from culture broth after mycelium removed. Lane 2: Crude proteins eluted from ceramic columns (3˜100 kDa). Lane 3: Crude proteins from 1^{st} step hydrophobic interaction chromatography. Lane 4: Crude proteins from 2^{nd} step Mono Q chromatography. Lane 5 and 6: Fractions (ScFz) were eluted from 3^{rd} step gel filtration chromatography with A280 nm absorbance 30.59 and 97.27.

FIGS. 3A and 3B show the Glycoprotein staining of ScFz applied on the SDS-PAGE, illustrating the SDS-PAGE stained with a CBR-250 (3A) and GelCode Glycoprotein Staining (3B), wherein “M” is the protein marker, Lane1 is the positive control of glycoprotein staining, Lane 2 is the negative control of glycoprotein staining, Lane 3 is the crude proteins from the culture broth after mycelium removed, and Lane 4 is the ScFz.

FIG. 4 is the fibrinogen degrading performance of purified fibrinolytic enzyme, ScFz, from S. commune culture broth on 10% SDS-PAGE, wherein “M” is Marker, Lane1˜7 are after incubation of ScFz with fibrinogen at 0, 0.5, 2, 6, 12, 22 and 30 hrs, α, β and γ, are the α-chain, β-chain and γ-chain of fibrinogen respectively, and the black arrows indicating the fibrinogen degraded products.

FIG. 5 shows the plasminogen activation performance of purified fibrinolytic enzyme, ScFz, from the S. commune culture broth, wherein the ScFz+PLG is 1 μg ScFz and plasminogen 0.0001 UN ( [symbol] ), the UK+PLG is 1 μg urokinase and plasminogen 0.0001 UN ( [symbol] ), the ScFz is 1 μg purified enzyme ( [symbol] ), the PL is the plasmin 1 μg ( [symbol] ), the PLG is plasminogen 0.0001 UN ( [symbol] ), and the 20 ug UK is 20 μg urokinase ( [symbol] )

FIG. 6 shows the fragment MS/MS plot, wherein the Mascot search results and peptide fragment view of ScFz protein identification (protein ID), wherein there is a MS/MS fragmentation of SGGGGGGGLGSGGSIR found in gi|81175178, Keratin, type I cytoskeletal 9 (Cytokeratin-9) (CK-9) (Keratin-9) (K9), Match to Query 160: 1231.683048 from (616.848800,2+) intensity(170.8776).

FIG. 7 shows the fibrinolytic activity assay of purified fibrinolytic enzyme, ScFz, from the S. commune culture broth compared to the human plasmin.

FIG. 8 is a table of purification and recovery of fibrinolytic enzyme, ScFz, from S. commune mycelium culture broth.

FIG. 9 is a table of N-terminal amino acid sequence of purified enzyme, ScFz, from S. commune mycelium culture broth compared to other fibrinolytic enzymes and metalloendopeptidases.

FIG. 10 is a table of fibrinolytic activity of purified enzyme, ScFz, from S. commune mycelium culture broth compared to human plasmin.

### DETAILED DESCRIPTION OF THE PREFERRED EMBODIMENT

As described herein, a novel fibrinolytic enzyme has been isolated from a mushroom, wherein the mushroom is cultured within fermentation in a culture broth, wherein an edible or medicinal mushroom is preferred for isolating the novel fibrinolytic enzyme, and especially preferred the mushroom called Schizophyllum commune. The novel fibrinolytic enzyme, which is a catalyst, is capable of degrading a fibrin or fibrinogen without activating a plasminogen.

The novel fibrinolytic enzyme, ScFz, isolated from the mushroom has at least one of the following characteristics (a) to (c): (a) the novel fibrinolytic enzyme has an amino acid N-terminus sequence of SEQ ID NO.1, ASYNGXSS, wherein A is alanine, S is serine, Y is tyrosine, N is asparagines, G is glycine, and X is undetermined, (b) the partial protein fragment of ScFz has the same protein fragment of the gil81175178 protein after the analyze of LC/MS/MS mass spectrographic method, and (c) the novel fibrinolytic enzyme has a molecular weight close to 23 kDa.

Accordingly, the novel fibrinolytic enzyme isolated from the culture broth of the mushroom is capable of degrading the fibrin which is one of the main compositions of a blood clot, wherein the blood clot may cause any thrombosis-related disease such as stroke or hypertension. Thus, the novel fibrinolytic enzyme can be used for the treatment of the thrombosis-related disease by directly degrading the fibrin or fibrinogen without activating the plasminogen to activate the plasmin to degrade the fibrin or fibrinogen, so as to prevent a side effect of hemorrhage due to the over activating the plasminogen to activate the plasmin as a conventional thrombolytic agent.

It is worth to mention that the conventional thrombolytic agent not only degrades the fibrin or fibrinogen but also activates the plasminogen to convert into the plasmin, such that the conventional thrombolytic agent of the prior arts usually risks a high danger of hemorrhage by over generating the plasmin.

The novel fibrinolytic enzyme is preferably isolated from a culture broth of the fermentation cultured Schizophyllum commune (S. commune), wherein the novel fibrinolytic enzyme is separated and purified from a secretion secreted by the mushroom in the culture broth. Accordingly, the novel plasmin isolated from the culture broth of the Schizophyllum commune is a monomer having the molecule weight 20˜23 kDa.

A process for producing a novel fibrinolytic enzyme from mushroom is provided, wherein the process comprises the steps of (a) culturing a mushroom by fermentation in a culture broth; (b) removing a mycelium of the mushroom from the above culture broth by filtration; (c) separating different molecular weight (MW) molecules of the culture broth by cross-flow filtration system using a ceramic columns, wherein the culture broth is separated into a lower molecular weight (MW) solution and a higher molecular weight solution; (d) precipitating a crude protein from the lower molecular weight solution by adding an ammonium sulfate and dialyzed; and (e) purifying the crude protein from the step (d) to get a fibrinolytic enzyme.

It is worth mentioning that the process for isolating the novel fibrinolytic enzyme can be applied on any types of mushroom, wherein an edible or medicinal mushroom is preferred, and especially preferred the mushroom called Schizophyllum commune.

In the step (a), the mushroom used for isolating the novel fibrinolytic enzyme is the mushroom called Schizophyllum commune, wherein the Schizophyllum commune is cultured in the culture broth of a liquid YM broth (Acumedia, USA), wherein the liquid YM broth of the culture broth has an enzymatic digestive gelatin, a malt extract, a dextrose, and a yeast extract, so that the mushroom of Schizophyllum commune cultured in the culture broth of the liquid YM broth is cultured at 28° C. with constant stirring and adequate air supply for 3 to 5 days.

In the step (b) of the process, after culturing the Schizophyllum commune mushroom, the mycelia of the Schizophyllum commune growing in the culture broth of liquid YM broth is removed by centrifugation at 8.23*1000 g for 30 minutes at 4° C., so as to precipitate the mycelia to get an upper limpid liquid of the culture broth for later use.

It is appreciated that in order to get a relatively more pure upper limpid liquid (supernatant of the culture broth after the centrifugation to remove the mycelia), the limpid liquid is further filtered by a 11 μm and a 2 μm filter paper with the exhaust filtering method respectively and sequentially, and then by a 0.45 μm Millipore membrane with pump filtering method to completely remove the mycelia, so that the relatively more pure limpid liquid of the culture broth of the Schizophyllum commune mushroom is gotten.

In the step (c) of the process, the limpid liquid of the culture broth of the Schizophyllum commune is further being separated into the lower molecular weight solution having the relatively lower molecular weight, and the higher molecular weight solution having the relatively higher molecular weight by the cross-flow system (Advanced Biotechnology Laboratories Co., R.O.C.) of filtration, so as to fractionate the limpid liquid of the culture broth, wherein a ceramic column is provided for the selection and fractionation the limpid liquid of culture broth to get a target protein.

In the present invention, a molecular weight exclusion limit of the ceramic column is around 50 to 150 kDa, better for using 80 to 120 kDa, and even better for using 100 kDa ceramic column for separating the limpid liquid of the culture broth, so that the molecular weight of a protein in the culture broth, which is larger than 100 kDa, is unable to penetrate the ceramic column, in such manner that the limpid liquid of the culture broth is separated into two liquids, so as to collect the target protein.

It is worth to mention that during the operation of the fractionation, a cycling pump is adapted for the assistance of the limpid liquid of the culture broth of the to circular in the fractionation system with a suitable flow rate flowing through the ceramic column and to cause a cross flow, so that the cross flow can ease the accumulation at the membrane. In order words, the cross flow can prevent the substance of the limpid liquid to block up the membrane, such that the cross-flow ceramic column can be more accurate for separating the proteins of the culture broth of the Schizophyllum commune.

When a circulating fluid of the limpid liquid of the culture broth goes through the ceramic column, the smaller molecules and water molecules can go through the ceramic column and become the lower molecular weight solution, wherein those larger molecules which are unable to pass through the ceramic column are staying in the circulating fluid and become the larger molecular weight solution. The lower molecular weight solution having the smaller molecules, which includes the target protein of the novel fibrinolytic enzyme, is usually diluted, and the larger molecular weight is the circulating fluid having the high concentration larger molecules.

It is appreciated that the lower molecular weight solution can be further filtered by another ceramic column such as a 3 kDa of the molecular weight exclusion limit of the ceramic column, such that the water molecules is removed to concentrate the lower molecular weight solution, so as to exclude the extreme small molecules to get a concentrated lower molecular weight solution of the limpid liquid of the culture broth.

In the step (d) of the process, a crude protein including the target protein is precipitated from the concentrated lower molecular weight solution, wherein the step (d) further comprises the steps of dialysis: (d-1) adding an ammonium sulfate to the concentrated lower molecular weight solution to 80% saturation with constant stirring at 4° C. overnight; (d-2) centrifuging the above concentrated lower molecular weight solution at 20,000 g at 4° C. for 30 minutes to obtain a first precipitation; (d-3) adding a secondary water to dissolve the first precipitation to form a precipitation fluid; (d-4) removing salt in the precipitation fluid by a dialysis against deionizer water at 4° C. for an additional 24 hours to obtain the crude protein.

It is worth mentioning that the ammonium sulfate is dried and grinded to powder so as to avoid excessive high concentration in local to cause the precipitation incomplete or uneven precipitation.

In the step (e), the purifying step of the crude protein, which has the target protein from the step (d), is processed by chromatography method, wherein the step (e) further comprises the following steps:

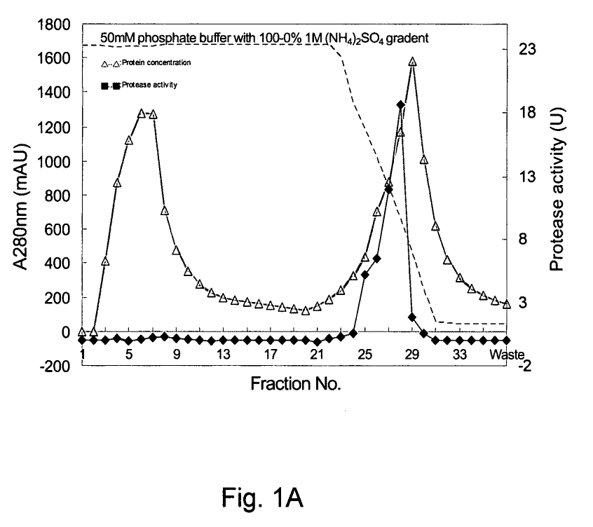

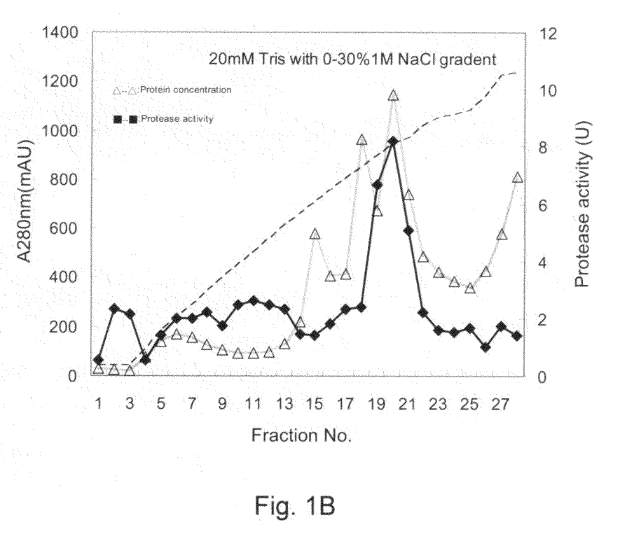

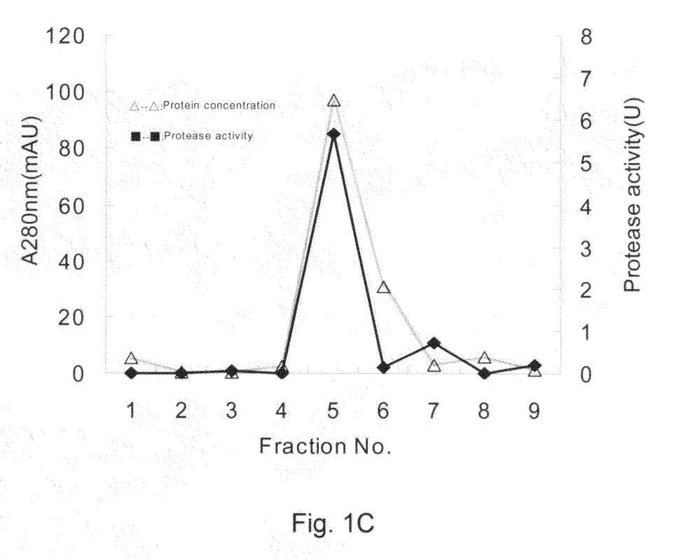

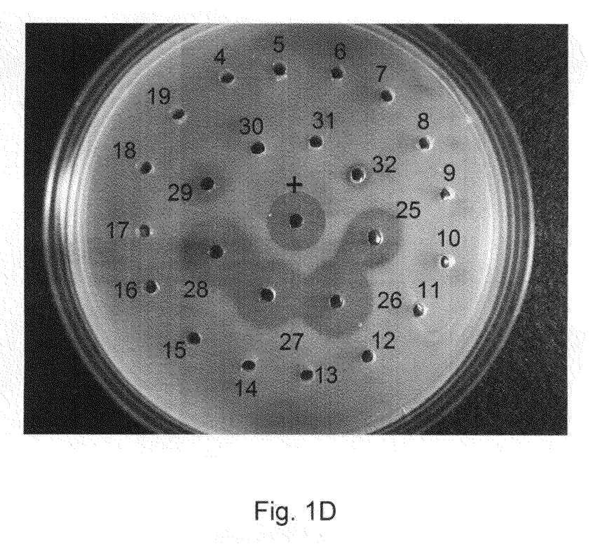

(e-1) a first step of chromatography, wherein a dialysis substrate from the step (d-4) is applied onto a first purification column (using the phenyl Sepharose™ high performance beaded packing column, provided by GE Healthcare Life Science) to chromatography the crude protein using the AKTA purifier 10 (Amersham Pharmacia Biotech) by hydrophobic interaction chromatography, which is previously equilibrated with 50 mM sodium phosphate buffer, wherein the sodium phosphate buffer has a Phosphoric acid solution of 1M (NH₄)₂SO₄ (pH=7) as start buffer at 1 ml/min flow rate. An eluent adapted as a mobile phase of the chromatography method has a gradient concentration (100-0%) 50 mM Phosphoric acid solution of 1M (NH₄)₂SO₄, and the flow rate of the eluent is set to 1 (ml/min) to elute the first purification column. After measuring a protease and a fibrinolytic activities for each elutes, a plurality of fractions, which is from each of the elutes, with fibrinolytic activity are collected, pooled, and precipitated by addition of ammonium sulfate and dialyzed in a 20 mM Tris buffer (pH=8). The result of the protein concentration and the protease activities of each elutes is shown in FIG. 1(A).

(e-2) a second step of chromatography, wherein a strong anion exchange chromatography is provided for the second step of chromatography, wherein after adjusting a sample of the chromatography experience of each fraction after the elutes to choose a first fraction for the second step of chromatography use according to a pH and ionic strength, the first fraction is applied onto a second purification column (Mono Q™ 5/50 GL column, GE Healthcare Life Science) with a flow rate of 1 ml/min and elute with a 20 fold bed volume of linear gradient to 0 to 30% 1M NaCl. The result of the protein concentration and the protease activities of each elutes is shown in FIG. 1(B).

(e-3) a third step of gel filtration, wherein the second fraction obtained from the second step of chromatography is applied onto a third purification column (Superdex 75 10/300 GL column. The result of the protein concentration and the protease activities of each elutes is shown in FIG. 1(C). 3962

It is worth to mention that the chromatography method applied for the purification process is capable for the performance of high purification rate, so that after the purification of chromatography, the relatively higher purified target protein from the crude protein is obtained, so as to get a relatively higher fibrinolytic activity.

Accordingly, the sample is further reacted with azocasein (Sigma), wherein the protease activity of the Schizophyllum commune novel fibrinolytic enzyme (ScFz) is determined by measuring the release of acid-soluble material after sample reacted from azocasein (Sigma). The ScFz sample fractions 100 μl is added to 100 μl 0.5% (W/V) azocasein solution, prepared in 0.1M sodium phosphate buffer, pH8.0, and then incubation at 37° C. for 15 minutes. After 15 minutes, a 350 μl of ice-cold 10% (W/V) trichloroacetic acid is added and mixed for another 10 minutes. After 10,000 g, 15 minutes of centrifugation at 4° C., a 500 μl supernatant therefrom is drew out and an equal volume of 0.5N NaOH is added into the supernatant. A quantity of acid-soluble material in the supernatant is measured by an absorbance at 440 nm, and the sample is used as a blank after boiling. One unit of protease activity is defined as acid-soluble material produced from the azocasein to yield an absorbance difference of 0.001 in the incubation at 37° C. for 15 minutes.

Accordingly, the protein concentration determination used in this present process for the fractions from each elutes is based on a Bradford method, Bio-Rad Protein Assay kit (Bio-Rad) for the protein concentration determination of the ScFz of the fractions. An acidic dye is added to the protein solution to allow 5 minutes reaction time and absorbance is measured at 595 nm with spectrophotometer (Ultrospec 1000). The protein concentration is determinate according to a standard curve that provides a relative measurement of bovine serum albumin (Sigma, USA).

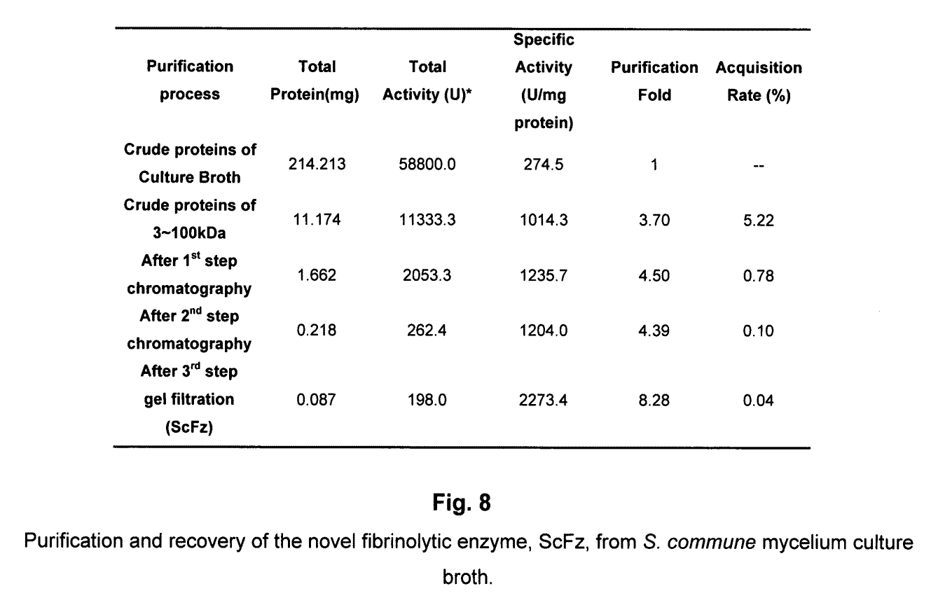

Accordingly, in order to measure a purification fold and yield of the fractions from the purifying process, the fractions of the product of each purification process step is measured to get the information of the total protein concentration, the protease activity, the total activity, so that a specific activity, a purification fold, and an acquisition rate as shown in FIG. 8, and can be calculated from the following formulas:

Specific Activity (U/mg)=protease activity (U)/total protein (mg)

Purification Fold=the fraction of product of each purification process step specific activity/broth protein content

Acquisition Rate=the fraction of production of each purification process step total protein/broth total protein content

It is worth mentioning that after three steps chromatography purification, the specific activity of ScFz is amplified to 8.28 fold of original culture broth.

It is appreciated that one unit of protease activity is defined as acid-soluble material produced from azocasein to yield 440 nm absorbance of 0.001 for 15 minutes incubation at 37° C.

Fibrinolytic and Fibrinogenolytic Performances:

The Fibrinolytic activity for the ScFz is determined using artificial fibrin plate assay and time course fibrinogen degradation reflected on SDS-PAGE. The Fibrin plate assay is modified from the method described by Astrup and Mullertz (1952), using 1.5% agarose, 0.2% human fibrinogen and 10 U human thrombin. The fibrin clots are made in a nine cm diameter Petri dish at room temperature, and apertures is made on the plate by air puncher of 3-4 mm diameter. A 20 μl of each eluted fraction is loaded in aperture on the plate and incubated for 24 hours at 20° C., then the diameter of the digestive circle is measured. The diameter is directly proportional to the potency of the fibrinolytic activity and a reagent, human plasmin, is used as positive control.

Plasminogen Activation Performances:

Plasminogen activation activity of ScFz is checked by using 0.1 mM plasmin specific substrate S2251 for plasminogen (SIGMA) activated reaction. A cleavage of the substrate, shown by generation of p-nitroaniline (pNA), is measured by following the initial increase in A405 at 30° C. and urokinase is used as positive control. To check the ScFz with the plasminogen activation with fibrin existence, plasminogen-rich and plasminogen-free artificial fibrin plate is then used and processed as described in the section of the Fibrinolytic Performance previously.

Molecular Weight Determination and Glycoprotein Staining:

A molecular weight determination of the target protein from the ScFz is determined by a SDS-PAGE for the ScFz with polyacrylamide gel and stained with Coomassie Brilliant Blue R-250 and GelCode Glycoprotein Staining Kit. For N-terminal sequence, PAGE is transferred on membrane and then sequenced. To estimate the native molecular weight, size exclusion is performed using Superdex 75 10/300 GL (GE Healthcare) high performance gel filtration at the flow rate of 1.2 ml/min with 50 mM phosphate buffer and 0.15M NaCl, pH 7.0. Protein markers including ribonuclease A (13.5 kDa), carbonic anhydrase (29 kDa), apoferritin (44.3 kDa) and bovine serum albumin (67 kDa) are used as standard.

Protein Identification (Protein ID):

After the SDS-PAGE running, the ScFz in gel is eluted, digested with trypsin, and then assayed by the Micromass Q-TOF ESI/MS/MS mass spectrometer. The result searched in MSACOT data base to looking for analogous protein.

N-Terminal Amino Acid Sequence:

After the SDS-PAGE running, it is transferred to a polyvinylidine difluoride (PVDF) membrane by electro blotting. The amino acids sequence of the protein is determined by Edman degradation method using an amino-acid sequencer (Applied Biosystems, Procise 494). Sequenced data and sequence alignment is analyzed by BLAST search in the NCBI protein database with default parameter.

Fibrinolytic Activity Compared to Human Plasmin:

According to the Fibrinolytic and Fibrinogenolytic Performances, the Schizophyllum fibrinolytic activity is examined by artificial fibrin plate assay compare with human plasmin. The digestive rings diameter is measured after treated with commercial human plasmin and the ScFz.

The result of the step (e) purifying of the process of producing novel fibrinolytic enzyme are shown in FIGS. 1A to 1C of the drawings, wherein after 80% of saturated ammonium sulfate precipitated, 11.2 mg of the crude proteins is obtained from 400 ml Schizophyllum commune (S. commune) culture broth. Fractions are separated and purified through consecutive chromatography with a hydrophobic interaction of the step (e-1), as shown in FIG. 1A, a strong anion exchange of the step (e-2) as shown in FIG. 1B, and a gel filtration of the step (e-3) as shown in FIG. 1C of the drawings. In the experiment performs three steps of purification. The protein concentration verse the protease activity is recorded as shown in FIGS. 1A to 1C. The significant protease activities are observed in fractions 25-28 from the Phenyl Sepharose™ High Performance column, in fractions 17-21 from the MonoQ column, and fraction 5 from the gel filtration column. The fraction from gel filtration column is then nominated as “ScFz”.

Reaction between fibrinolytic activities of each fractions obtains from (e-1) step purification column against an artificial fibrin plate is showed in FIG. 1D. A visible digestive zone is observed from fractions 25-28. Therefore, the protease activity of this experiment, performed on MonoQ and gel filtration columns, is the criteria for the purification procedures. The final novel fibrinolytic enzyme specific activity increases 8.28 fold, from the original culture broth of 274.5 U/mg protein to 2273.4 U/mg protein, as the steps in purification of ScFz is summarized in FIG. 8. One unit of protease activity is defined as the acid-soluble material produced from azocasein to yield 440 nm absorbance of 0.001 during incubation at 37° C. within 15 minutes.

Molecular Weight Determination and Glycoprotein Staining:

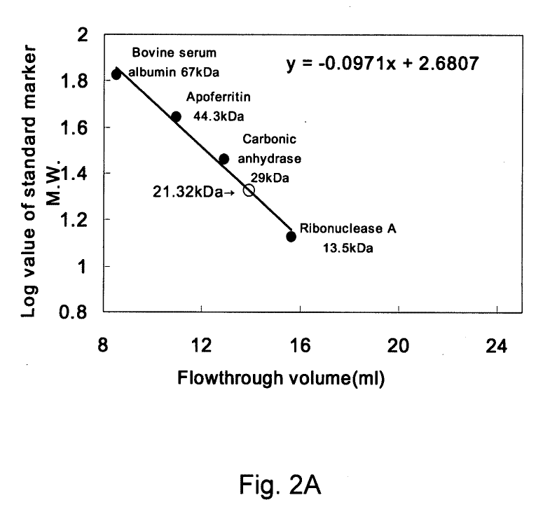

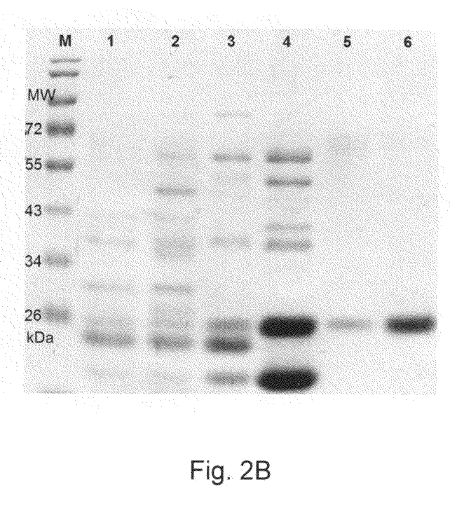

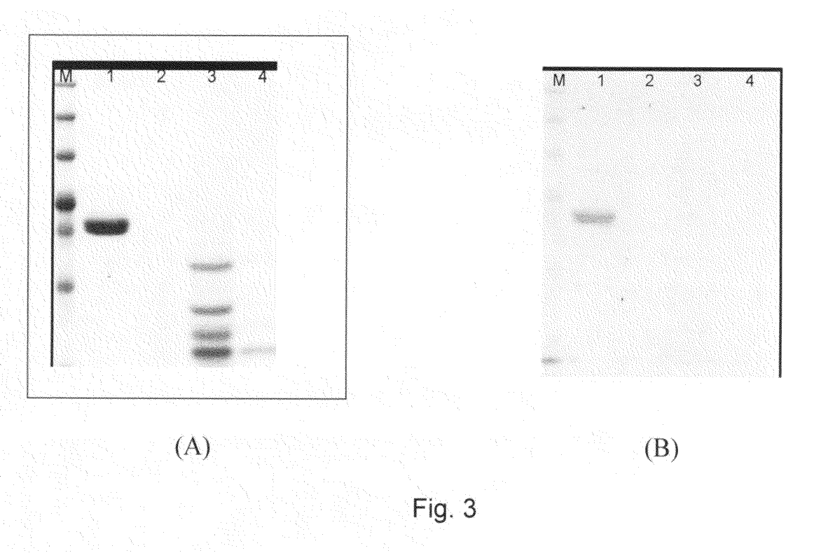

In this experiment, the novel fibrinolytic enzyme, ScFz, from S. commune culture broth is isolated. The molecular weight, under reducing and non-reducing condition by gel filtration on the Superdex 75 10/300 GL column (FIG. 2A) and SDS-PAGE (FIG. 2B), is estimated to be 21.33 kDa and revealed a purified single band. Result obtained from glycoprotein staining, shows a negative response, as shown in FIG. 3, indicating that the ScFz is a non-carbohydrate molecular.

Fibrinolytic and Fibrinogenolytic Performances:

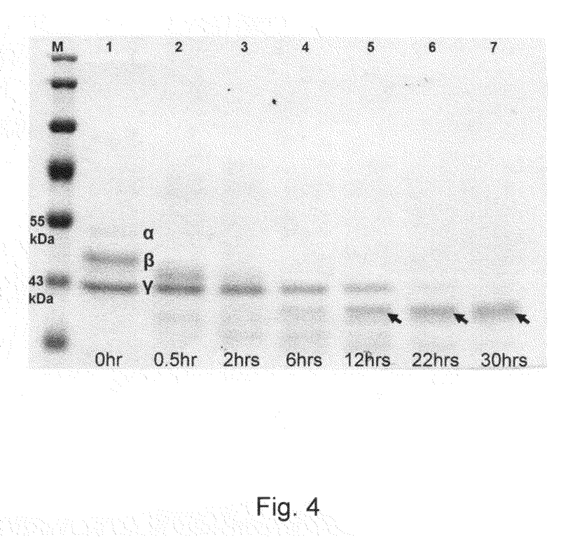

In an artificial fibrin plate assay, the fibrinolytic enzyme purified from S. commune crude proteins containing fibrinolyitc activity resulted in visible transparent region on agarose plate formed by digest artificial fibrin (FIG. 1D). Pattern of fibrinogen degradation by the ScFz is also analyzed by the SDS-PAGE. The results obtained from this experiment show an obvious degradation of fibrinogen α-chain and fibrinogen β-chain after 30 minutes, and completely degradation of γ-chain within 22 hours after incubation. The experiment also shows that at 30 hours after incubation, fibrinogen degradation for ScFz continuously exist and fibrinogen degraded products are significantly observed in 12 to 30 hours after incubation as shown in FIG. 4, black arrows.

Plasminogen Activation Performances:

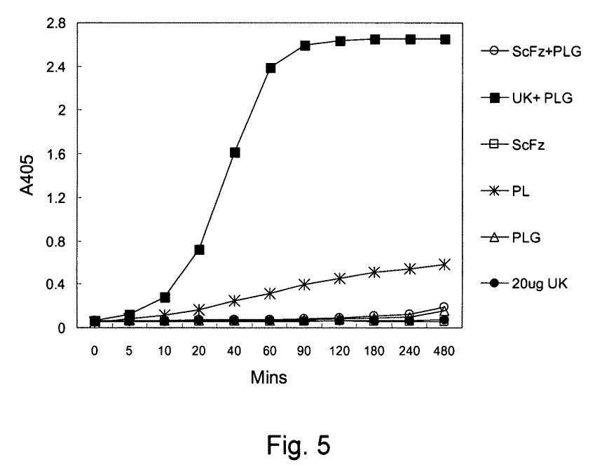

Plasminogen activation activity for ScFz is determined to verify the presence of fibrin. The results obtained from plasminogen-rich artificial fibrin plate test shows that there is no effect of enzyme activity over the plasminogen when fibrin exist. In plasmin specific substrate S2251 reaction, the ScFz incubated with plasminogen without fibrin does not convert the plasminogen to plasmin. In comparison, the positive control (urokinase) shows a significant increasing of plasmin in the tested system in 405 nm absorbance as shown in FIG. 5.

Protein Identification (Protein ID):

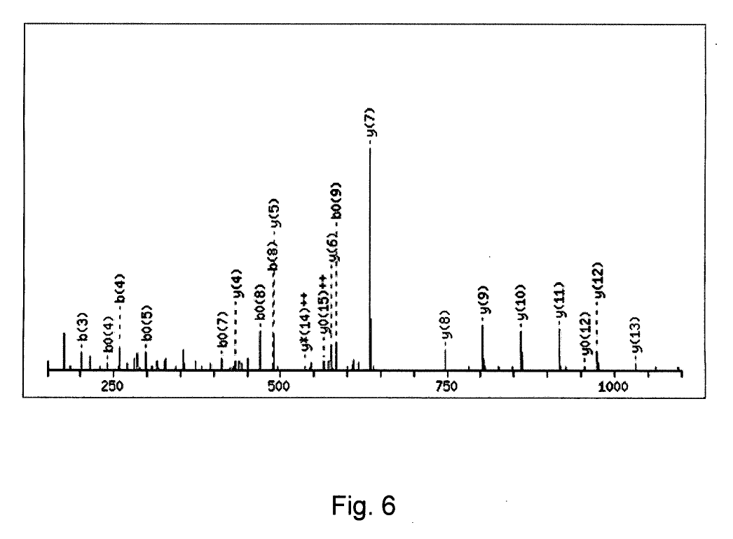

Referring to FIG. 6 of the drawings, there is a MS/MS fragmentation of SGGGGGGGLGSGGSIR, which is found in gi|81175178 Keratin, type I cytoskeletal 9 (Cytokeratin-9) (CK-9) (Keratin-9) (K9). The result indicates that the ScFz is a novel protein that compare to presently known protein.

N-Terminal Amino Acid Sequence:

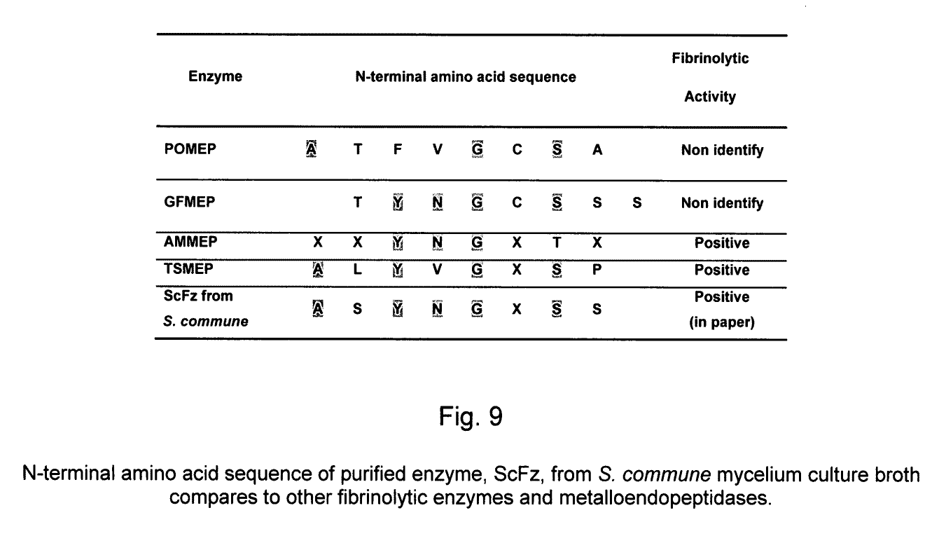

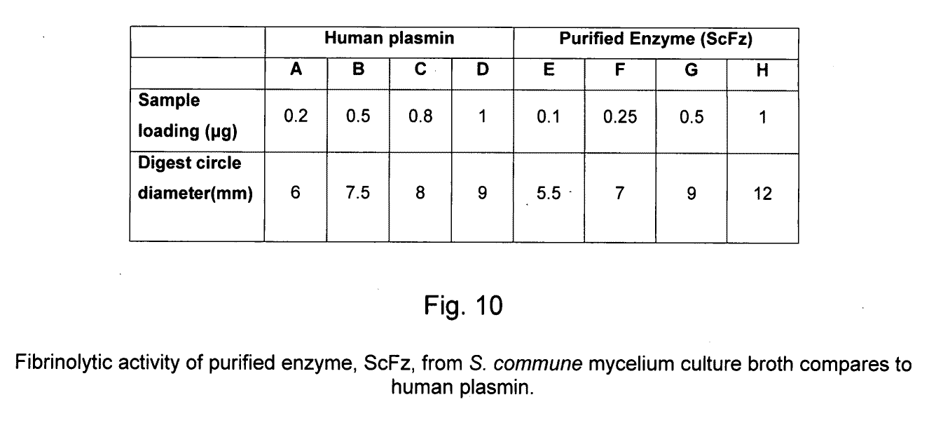

The first eight amino acid residues of the ScFz are demonstrated to be ASYNGXSS (X means amino acid undetermined). The sequence is slightly different compared to metalloendopeptidases (MEPs) from Grifola frondosa, Pleurotus ostreatus, Armillariella mellea and Tricholoma saponaceum (Nonaka et al., 1997; Kim and Kim, 1999; Kim and Kim, 2001) as presented in FIG. 9.

Fibrinolytic Activity Compared to Human Plasmin:

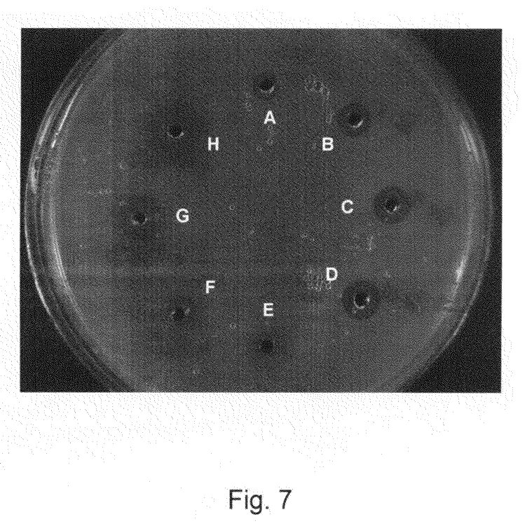

As the result showed in FIGS. 7 and 10, digest circle diameter is measured after experiment, and the zone increases as concentration increasing. The ScFz makes the equal digestive zone on fibrin plate with human plasmin while needs the twice dosage. Therefore, the high fibrinolytic activity of ScFz is expected for apply to be the thrombolytic agents for present thrombosis related diseases.

It is appreciated that a conventional method for separating an enzyme from the mushroom is to use an aqueous solution, organic solvent, or other physical technology to extra the enzyme from a carpophores or mycelium of the mushroom. It is not only time consuming but also involving complicated steps. Also, a plurality of specific devices is required for the conventional method to purify the enzyme such as the plasmin. Thus, to isolate the novel plasmin of the enzyme of the mushroom directly from the secretion of the mushroom of a liquid medium, such as the culture broth, not only simplify the process to isolate the enzymes of the mushroom but also get a relatively higher activity of the enzymes.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A process of producing fibrinolytic enzyme ScFz, comprising the steps of:
(a) providing a culture broth to culture a mushroom therein;
(b) removing a mycelium of said mushroom from said culture broth by a filtration, wherein an upper limpid liquid of said culture broth is obtained;
(c) separating a plurality of different molecular weights molecules of said limpid liquid of said culture broth, wherein said culture broth is separated into a lower molecular weight solution and a higher molecular weight solution;
(d) precipitating a crude protein from said lower molecular weight solution; and
(e) purifying said crude protein precipitated from said step (d) to get a target protein of said fibrinolytic enzyme from said mushroom.

2. The process, as recited in claim 1, wherein said mushroom is a Schizophyllum commune.

3. The process, as recited in claim 1, wherein before said step (b) further comprises a step of centrifuging said culture broth to get a limpid liquid of said culture broth.

4. The process, as recited in claim 2, wherein before said step (b) further comprises a step of centrifuging said culture broth to get a limpid liquid of said culture broth.

5. The process, as recited in claim 1, wherein a cycling pumping of a cross-flow system is provided for separating said culture broth into said lower and said higher molecular weight solution.

6. The process, as recited in claim 4, wherein a cycling pumping of a cross-flow system is provided for separating said culture broth into said lower and said higher molecular weight solution.

7. The process, as recited in claim 1, wherein at least one ceramic column is applied on said cross flow system as a molecular weight exclusion limit for separating said culture broth into said lower and said higher molecular weight solution, wherein said molecular weight exclusion limit of said ceramic column has a range between 50 and 150 kDa.

8. The process, as recited in claim 6, wherein at least one ceramic column is applied on said cross flow system as a molecular weight exclusion limit for separating said culture broth into said lower and said higher molecular weight solution, wherein said molecular weight exclusion limit of said ceramic column has a range between 50 and 150 kDa.

9. The process, as recited in claim 7, wherein a 3 kDa molecular weight exclusion limit of said ceramic column is further applied on said lower molecular weight solution for filtering a plurality of H₂O molecules to concentrate said lower molecular weight solution.

10. The process, as recited in claim 8, wherein a 3 kDa molecular weight exclusion limit of said ceramic column is further applied on said lower molecular weight solution for filtering a plurality of H₂O molecules to concentrate said lower molecular weight solution.

11. The process, as recited in claim 1, wherein said step (d) further comprises a step of removing salt in said crude protein by dialysis.

12. The process, as recited in claim 6, wherein said step (d) further comprises a step of removing salt in said crude protein by dialysis.

13. The process, as recited in claim 10, wherein said step (d) further comprises a step of removing salt in said crude protein by dialysis.

14. The process, as recited in claim 1, wherein, in said step (e), said crude protein is purified to get relatively more purified said target protein from said crude protein by chromatography method.

15. The process, as recited in claim 6, wherein, in said step (e), said crude protein is purified to get relatively more purified said target protein from said crude protein through chromatography method.

16. The process, as recited in claim 13, wherein, in said step (e), said crude protein is purified to get relatively more purified said target protein from said crude protein through chromatography method.

17. The process, as recited in claim 14, wherein said chromatography method in said step (e) comprises the steps of:
(e-1) applying said crude protein from said step (d) onto a first purification column by hydrophobic interaction chromatography, wherein a first fraction from a plurality of elutes of said hydrophobic interaction is selected for a next step;
(e-2) applying said first fraction of said crude protein from said step (e-1) onto a second purification column by strong anion exchange chromatography, wherein a second fraction of said anion exchange chromatography after a plurality of elutes of said anion exchange chromatography is selected for a next step; and
(e-3) applying said second fraction of said crude protein from said step (e-2) onto a third purification column by gel filtration of chromatography, wherein said second fraction is applied onto said third purification column to get a third fraction selected from a plurality of elutes from said third purification column, so as to get said target protein of said novel fibrinolytic enzyme.

18. The process, as recited in claim 15, wherein said chromatography method in said step (e) comprises the steps of:
(e-1) applying said crude protein from said step (d) onto a first purification column by hydrophobic interaction chromatography, wherein a first fraction from a plurality of elutes of said hydrophobic interaction is selected for a next step;
(e-2) applying said first fraction of said crude protein from said step (e-1) onto a second purification column by strong anion exchange chromatography, wherein a second fraction of said anion exchange chromatography after a plurality of elutes of said anion exchange chromatography is selected for a next step; and
(e-3) applying said second fraction of said crude protein from said step (e-2) onto a third purification column by gel filtration of chromatography, wherein said second fraction is applied onto said third purification column to get a third fraction selected from a plurality of elutes from said third purification column, so as to get said target protein of said novel fibrinolytic enzyme.

19. The process, as recited in claim 16, wherein said chromatography method in said step (e) comprises the steps of:
(e-1) applying said crude protein from said step (d) onto a first purification column by hydrophobic interaction chromatography, wherein a first fraction from a plurality of elutes of said hydrophobic interaction is selected for a next step;
(e-2) applying said first fraction of said crude protein from said step (e-1) onto a second purification column by strong anion exchange chromatography, wherein a second fraction of said anion exchange chromatography after a plurality of elutes of said anion exchange chromatography is selected for a next step; and
(e-3) applying said second fraction of said crude protein from said step (e-2) onto a third purification column by gel filtration of chromatography, wherein said second fraction is applied onto said third purification column to get a third fraction selected from a plurality of elutes from said third purification column, so as to get said target protein of said novel fibrinolytic enzyme.

20. A fibrinolytic enzyme, ScFz, having a molecular weight around 20 to 23 kDa and comprising an amino acid N-terminus sequence of SEQ ID NO.1, ASYNGXSS, wherein A is alanine, S is serine, Y is tyrosine, N is asparagines, G is glycine, and X is undetermined, wherein said fibrinolytic enzyme is adapted for degrading a fibrin and a fibrinogen without activating plasminogen to plasmin.

21. The fibrinolytic enzyme, as recited in claim 20, which has a partial protein fragment the same as “gil81175178” protein fragment according to a LC/MS/MS mass spectrographic analysis.

22. The fibrinolytic enzyme, as recited in claim 21, which is isolated from a mushroom by the step of:
(a) providing a culture broth to culture said mushroom therein;
(b) removing a mycelium of said mushroom from said culture broth by a filtration, wherein an upper limpid liquid of said culture broth is obtained;
(c) separating a plurality of different molecular weights molecules of said limpid liquid of said culture broth, wherein said culture broth is separated into a lower molecular weight solution and a higher molecular weight solution;
(d) precipitating a crude protein from said lower molecular weight solution; and
(e) purifying said crude protein precipitated from said step (d) to get a target protein of said fibrinolytic enzyme from said mushroom.

23. The fibrinolytic enzyme, as recited in claim 22, wherein said mushroom is Schizophyllum commune.

24. The fibrinolytic enzyme, as recited in claim 21, which is isolated from a culture broth of a mushroom, wherein said culture broth contains a secretion from said mushroom.

25. The fibrinolytic enzyme, as recited in claim 24, wherein said mushroom is Schizophyllum commune.
